SEQUENCE LISTING (48 sequences)

SEQ ID NO: 1 - DNA, 939 bp, Neisseria meningitidis
  CDS (1)..(939)
atgtccatccatactctgaaacgcctgccctcatcgctgctgctcggtctctgcctttccctgccgtcagcccacctttttgccgacaacgacattttagggcaatttttagaacagaacatgcttacctcctccgatccgatagaaatattcgccgaaagcacgatacaccccaccaacacccaagccattacaggcggtctgattctctcctcacagtctgccctggtcgtcaacaacaaaaccggacagatactgtatcagaaaaacgccgacaggattatgcccatcgcctccatttccaaactgatgagcgcgatggtcgttttggatgcaaacttggacatgaacgaaaccgttaccattacgcccgacgaaatcgaccgcatcaaagggaccggcagccgtcttgccataggtacggcacttacacgcaaaaaactgctgcacctgagcctgatgagcagcgaaaaccgcgccacccatgcattgggcagaacctaccccggcggcatgggcgcatttgtcgccgccatgaaccgcaaagcccaaagcctcggtatgtacggcagccgcttttacgaaccgaccggactcaacttccaaaacgtttctaccgccaaagacctgagccttatggtcaacgccgccgcccaatatccgcaaatccgcaccaactcgacttccaactacgcctcggtacagaccaaaaacgggcagcagaactacaaaaactccaatgccctggtcagagaaggcatgtggaacatcgaattgcagaaaaccggctacatacgcgaagcaggcaggtctatggttgtcaaagccaacattcaaaaccaacccgttaccatcgtattgctgaactcgcccacatccgccacacgcgtcaacgacgcccgcaaaatcgaatcgtggatgctgcagcaacgctcctga

SEQ ID NO: 2 - protein, 312 aa, Neisseria meningitidis
MSIHTLKRLPSSLLLGLCLSLPSAHLFADNDILGQFLEQNMLTSSDPIEIFAESTIHPTNTQAITGGLILSSQSALVVNNKTGQILYQKNADRIMPIASISKLMSAMVVLDANLDMNETVTITPDEIDRIKGTGSRLAIGTALTRKKLLHLSLMSSENRATHALGRTYPGGMGAFVAAMNRKAQSLGMYGSRFYEPTGLNFQNVSTAKDLSLMVNAAAQYPQIRTNSTSNYASVQTKNGQQNYKNSNALVREGMWNIELQKTGYIREAGRSMVVKANIQNQPVTIVLLNSPTSATRVNDARKIESWMLQQRS

SEQ ID NO: 3 - DNA, 1239 bp, Neisseria meningitidis
  CDS (1)..(1239)
atggctttttatgcttttaaggcgatgcgtgcggccgcgttggctgccgccgttgcattggtactgtcgtcttgcggtaaaggcggagacgcggcgcagggcgggcagcctgctggtcgggaagcccctgcgcccgtcgtcggtgtcgtaaccgtccatccgcaaaccgtcgcattgaccgtcgagttgccggggcgtttggaatcgctgcgtaccgccgatgtccgcgcccaagtcggcggcatcatccaaaaacgcctgttccaagaaggcagttatgtccgtgccggacagccgctgtatcagatcgacagttccacttatgaagcaggtctggaaagcgcgcgcgcgcaactggcaacggctcaggcaacgcttgccaaagcggatgcggatttggcgcgatacaagcctttggttgccgccgaagccgtcagccggcaggaatacgatgctgcggtaacggcgaaacgttctgccgaggcaggcgttaaagcggcgcaggcggcaatcaaatccgccggcatcagcctgaaccgttcgcgcattaccgcgccgatttccggctttatcggtcagtccaaagtttccgaaggtacgttgctgaacgctggcgatgcgaccgtactggcgaccatccgccaaaccaatccgatgtatgtgaacgttacccagtctgcatccgaagtgatgaaattgcgccgtcagatagccgaaggcaaactgctggcggcggatggtgtgattgcggtcggcatcaaatttgacgacggcacagtttaccctgaaaaaggccgcctgctgtttgccgatccggccgtcaacgaatcgaccggtcagattaccctgcgcgccgccgtaccgaacgatcagaatatcttgatgcccggtctgtatgtgcgcgtgctgatggaccaagtggcggtggataacgcatttgttgtgccgcagcaggcggtaacgcgcggtgcgaaagataccgtgatgattgtgaatgcccaaggcggtatggaaccccgcgaggtaacggttgcgcaacagcagggtacgaattggattgttacgtcgggtctgaaggacggggacaaggtggttgtggaaggcatcagtatcgccggtataacgggtgcgaaaaaggtaacgcccaaagaatgggcgtcgtctgaaaaccaagccgccgcgcctcaatccggcgttcagacggcatctgaagccaaacctgcttctgaagcgaaataa

SEQ ID NO: 4 - protein, 412 aa, Neisseria meningitidis
MAFYAFKAMRAAALAAAVALVLSSCGKGGDAAQGGQPAGREAPAPVVGVVTVHPQTVALTVELPGRLESLRTADVRAQVGGIIQKRLFQEGSYVRAGQPLYQIDSSTYEAGLESARAQLATAQATLAKADADLARYKPLVAAEAVSRQEYDAAVTAKRSAEAGVKAAQAAIKSAGISLNRSRITAPISGFIGQSKVSEGTLLNAGDATVLATIRQTNPMYVNVTQSASEVMKLRRQIAEGKLLAADGVIAVGIKFDDGTVYPEKGRLLFADPAVNESTGQITLRAAVPNDQNILMPGLYVRVLMDQVAVDNAFVVPQQAVTRGAKDTVMIVNAQGGMEPREVTVAQQQGTNWIVTSGLKDGDKVVVEGISIAGITGAKKVTPKEWASSENQAAAPQSGVQTASEAKPASEAK

SEQ ID NO: 5 - DNA, 936 bp, Neisseria meningitidis
  CDS (1)..(936)
atggaactctttaaaatcaaacgcgatattccgtttatgagctacggcaaactgacgaccttcatttcgttggttacgtttatcgctgccgtgttctttttggttaccagaggtctgaatttctctgtcgaatttaccggcggtacggtaatggaagtccaatatcagcagggtgcggatgtcaataagatgcgcgaacgcctcgatacgctgaaaataggtgatgtacaggttcaggcattgggtacgaacaaacacatcatgatccgcctgccgaacaaagaaggtgttacttccgcacagttgtccaatcaggttatggatttgctgaaaaaagacagtcccgacgttaccttgcgccaagtcgaatttatcggcccgcaagtcggtgaggaattggtaagtaatggattgatggctttaggttttgtcgttatcggcatcattatttacctgtcgatgcgttttgaatggcgttttgccgtatctgccattatcgccaatatgcacgacatcgtgattattctcggctgctttgccttcttccaatgggaattttcgctgaccgtcttggcgggtatccttgccgtattgggctattctgtgaacgaatccgtcgtcgtcttcgaccgtatccgtgaaaacttccgcaagccggcgatgcgcggacatgccgtgccggaagtcatcgacaacgcgattaccgcaacgatgagccgcaccatcattacccacggttcgaccgaggcgatggtcgtatccatgctggtgttcggcggtgcggccttgcacggcttttctatggcgttgaccattggcatcgtgttcggcatttattcttccgtattggttgccagcccgcttctgctaatgttcggtttgagccgcgacaatatcggtaaagaaccgaagaagaaagaagaaatcgtggtttga

SEQ ID NO: 6 - protein, 311 aa, Neisseria meningitidis
MELFKIKRDIPFMSYGKLTTFISLVTFIAAVFFLVTRGLNFSVEFTGGTVMEVQYQQGADVNKMRERLDTLKIGDVQVQALGTNKHIMIRLPNKEGVTSAQLSNQVMDLLKKDSPDVTLRQVEFIGPQVGEELVSNGLMALGFVVIGIIIYLSMRFEWRFAVSAIIANMHDIVIILGCFAFFQWEFSLTVLAGILAVLGYSVNESVVVFDRIRENFRKPAMRGHAVPEVIDNAITATMSRTIITHGSTEAMVVSMLVFGGAALHGFSMALTIGIVFGIYSSVLVASPLLLMFGLSRDNIGKEPKKKEEIVV

SEQ ID NO: 7 - DNA, 2277 bp, Neisseria meningitidis
  CDS (1)..(2277)
gtgtccgaacccatacagcctaccagcctgagcctcggttcgacctgcctgttttgcagtaacgaaagcggcagccccgagagaaccgaagccgccgtccaaggcagcggcgaagcatccatccccgaagactatacgcgcattgttgccgacaggatggaaggacagtcgcaggtgcaggtgcgtgccgaaggcaacgtcgtcgtcgaacgcaaccggacgaccctcaataccgattgggcggattacgaccagtcgggcgacaccgttaccgcaggcgaccggttcgccctccaacaggacggtacgctgattcggggcgaaaccctgacctacaatctcgagcagcagaccggggaagcgcacaacgtccgcatggaaatcgaacaaggcggacggcggctgcaaagcgtcagccgcaccgccgaaatgttgggcgaagggcattacaaactgacggaaacccaattcaacacctgttccgccggcgatgccggctggtatgtcaaggcagcctctgtcgaagccgatcgggaaaaaggcataggcgttgccaaacacgccgccttcgtgttcggcggcgttcccattttctacaccccttgggcggacttcccgcttgacggcaaccgcaaaagcggcctgcttgttccctcactgtccgccggttcggacggcgtttccctttccgttccctattatttcaaccttgcccccaatctcgatgccacgttcgcgcccagcgtgatcggcgaacgcggcgcggtctttgacgggcaggtacgctacctgcggccggattatgccggccagtccgacctgacctggctgccgcacgacaagaaaagcggcaggaataaccgctatcaggcgaaatggcagcatcggcacgacatttccgacacgcttcaggcgggtgtcgatttcaaccaagtctccgacagcggctactaccgcgacttttacggcaacaaagaaatcgccggcaacgtcaacctcaaccgccgtgtatggctggattatggcggcagggcggcgggcggcagcctgaatgccggcctttcggttctgaaataccagacgctggcaaaccaaagcggctacaaagacaaaccgtatgccctcatgccgcgcctttcggtcgagtggcgtaaaaacaccggcagggcgcaaatcggcgtgtccgcacaatttacccgattcagccacgacagccgccaagacggcagccgcctggtcgtctatcccgacatcaaatgggatttcagcaacagctggggctatgtccgtcccaaactcggactgcacgccacctattacagcctcaaccgcttcggcagccaagaagcccgacgcgtcagccgcactctgcccattgtcaacatcgacagcggcgcaacttttgagcggaatacgcggatgttcggcggagaagtcctgcaaaccctcgagccgcgcctgttctacaactatattcctgccaaatcccaaaacgacctgcccaatttcgattcgtcggaaagcagcttcggctacgggcagctctttcgcgaaaacctctattacggcaacgacaggattaacaccgcaaacagcctttccgccgccgtgcaaagccgtattttggacggcgcgacgggggaagagcgtttccgcgccggcatcggtcagaaattctatttcaaggatgatgcggtgatgcttgacggcagcgtcggcaaaaaaccgcgcaaccgttccgactgggtggcatttgcctccggcagcatcggcagccgcttcatcctcgacagcagcatccactacaaccaaaacgacaaacgcgccgagaactacgccgtcggtgcaagctaccgtcccgcacagggcaaagtgctgaacgcccgctacaaatacgggcgcaacgaaaaaatctacctgaagtccgacggttcctatttttacgacaaactcagccagctcgacctgtccgcacaatggccgctgacgcgcaacctgtcggccgtcgtccgttacaactacggttttgaagccaaaaaaccgatagaggtgctggcgggtgcggaatacaaaagcagttgcggctgctggggcgcgggcgtgtacgcccaacgctacgttaccggcgaaaacacctacaaaaacgctgtctttttctcacttcagttgaaagacctcagcagtgtcggcagaaaccccgcagacaggatggatgtcgccgttcccggctatatcaccgcccactctctttccgccggacgcaacaaacgaccctga

SEQ ID NO: 8 - protein, 758 aa, Neisseria meningitidis
VSEPIQPTSLSLGSTCLFCSNESGSPERTEAAVQGSGEASIPEDYTRIVADRMEGQSQVQVRAEGNVVVERNRTTLNTDWADYDQSGDTVTAGDRFALQQDGTLIRGETLTYNLEQQTGEAHNVRMEIEQGGRRLQSVSRTAEMLGEGHYKLTETQFNTCSAGDAGWYVKAASVEADREKGIGVAKHAAFVFGGVPIFYTPWADFPLDGNRKSGLLVPSLSAGSDGVSLSVPYYFNLAPNLDATFAPSVIGERGAVFDGQVRYLRPDYAGQSDLTWLPHDKKSGRNNRYQAKWQHRHDISDTLQAGVDFNQVSDSGYYRDFYGNKEIAGNVNLNRRVWLDYGGRAAGGSLNAGLSVLKYQTLANQSGYKDKPYALMPRLSVEWRKNTGRAQIGVSAQFTRFSHDSRQDGSRLVVYPDIKWDFSNSWGYVRPKLGLHATYYSLNRFGSQEARRVSRTLPIVNIDSGATFERNTRMFGGEVLQTLEPRLFYNYIPAKSQNDLPNFDSSESSFGYGQLFRENLYYGNDRINTANSLSAAVQSRILDGATGEERFRAGIGQKFYFKDDAVMLDGSVGKKPRNRSDWVAFASGSIGSRFILDSSIHYNQNDKRAENYAVGASYRPAQGKVLNARYKYGRNEKIYLKSDGSYFYDKLSQLDLSAQWPLTRNLSAVVRYNYGFEAKKPIEVLAGAEYKSSCGCWGAGVYAQRYVTGENTYKNAVFFSLQLKDLSSVGRNPADRMDVAVPGYITAHSLSAGRNKRP

SEQ ID NO: 9 - DNA, 939 bp, Neisseria meningitidis; identical to SEQ ID NO: 1
  CDS (1)..(939)

SEQ ID NO: 10 - protein, 312 aa, Neisseria meningitidis; identical to SEQ ID NO: 2

SEQ ID NO: 11 - DNA, 1806 bp, Neisseria meningitidis
  CDS (1)..(1806)
atgaaaaaactgatttgcctgttcgccgtatttttgatgttgtgcggacgagctttcgcgctggatgcgaacgatctgctgccgccggaaaaggcattcgtgccggagcttgccgttgccgacgacggtgtgaacgtccgtttcaggattgccgacggatactatatgtatcaggcgaaaatcgtcggcaagaccgatccggcggatttgttgggacagccttctttcagcaagggcgaagagaaggaagacgagtttttcggcaggcagacggtttaccatcacgaggcgcaggttgcctttccttatgcaaaggctgtcggcgaaccgtataaattggttttgacctatcagggctgtgccgaagccggcgtgtgctatccgcccgtggataccgagtttgatattttcggcaacggcacttaccatccgcaaaccgacgaaccggcatccgccaaagaccgctttttgcagccttcctctcaaaacggcagcggggcgctgccgcccccgaagggggatgagggcggcgacagccgtttcaagctgtcttgggatacgctcaacgccaatcttttggcgttttttctcgctggtttgggcctgagttttaccgcctgtatgtatcccctgttgccgattgtttccagtattgtggtcggcgacaaaaaggcgggcaaggcgcgggcgtttgtgctgtccgtcgtttatgttcagggtttggctctgacttatacgctggtcggcattgttgccggactgacgggcgcactgctgaccgtatggttgcagcaggcttgggtggtattggcggcatcggctttaatggtcgtcttggcactgtctatgttcgggctgttcaacatccagcttcccaacgccgtgcagtcgtattttcagaatcaaagcagcaggctttcaggcggtaaaatcgtttccgtctttattatgggcatattgtccgcgctgattgtcgggccgtgcgtcgccccgccgctggcatttgctttgggctacatcggtcagacgggcgatgcggttttaggcggtttggcactttacactttggcgttgggcaccggcgttccgctgattgccatcggcacgttcggcgggcatatcctgcctaaggcaggcgattggatgaatgccgtcaaatacgcattcggcttcatcctgctagccgtcgccgtttacctcgccacgccgcacttgccctattatctcgtcgtcgcgctgtacacgctgctgatgctggttcctgcctttatgctgctggtcaacggacgcaggcagaaacgccgtccgaaagctgtggcattcgcattgggcggtatattgctgataggcggcgcgtggttcggctggcagggcgcaaacggcaaaacgaccgcgctgcaccatttcctgaccctcaatccaccagccgaagcaggcaaatcttcggaacacggcaaaatgtttgccgatactgccgcgctgaaggcagcgatggatacggcgttgaaagaacatcccgacaaacccgtcgttttggatttttatgccgactggtgcatttcctgcaaagaaatggcggcttacacgctcaatcagccggaagtgcatcaggcagtcgatatggaacgctttttccaaatcgacgtaaccgccaacacgcccgaacatcaggcgttgttgaaagaatacggtctgttcgggccgccgggcgtgtttgtcgtccgctccgacggcagccgcagcgagccgctgctgggttttgtcaaagcagacaagtttatcgagtggtatgaacaaaaccgctga

SEQ ID NO: 12 - protein, 601 aa, Neisseria meningitidis
MKKLICLFAVFLMLCGRAFALDANDLLPPEKAFVPELAVADDGVNVRFRIADGYYMYQAKIVGKTDPADLLGQPSFSKGEEKEDEFFGRQTVYHHEAQVAFPYAKAVGEPYKLVLTYQGCAEAGVCYPPVDTEFDIFGNGTYHPQTDEPASAKDRFLQPSSQNGSGALPPPKGDEGGDSRFKLSWDTLNANLLAFFLAGLGLSFTACMYPLLPIVSSIVVGDKKAGKARAFVLSVVYVQGLALTYTLVGIVAGLTGALLTVWLQQAWVVLAASALMVVLALSMFGLFNIQLPNAVQSYFQNQSSRLSGGKIVSVFIMGILSALIVGPCVAPPLAFALGYIGQTGDAVLGGLALYTLALGTGVPLIAIGTFGGHILPKAGDWMNAVKYAFGFILLAVAVYLATPHLPYYLVVALYTLLMLVPAFMLLVNGRRQKRRPKAVAFALGGILLIGGAWFGWQGANGKTTALHHFLTLNPPAEAGKSSEHGKMFADTAALKAAMDTALKEHPDKPVVLDFYADWCISCKEMAAYTLNQPEVHQAVDMERFFQIDVTANTPEHQALLKEYGLFGPPGVFVVRSDGSRSEPLLGFVKADKFIEWYEQNR

SEQ ID NO: 13 - DNA, 1857 bp, Neisseria meningitidis
  CDS (1)..(1857)
atgatgaaccgttatcctttatggaaatatctgctgattgtgttcacgattgcggttgccgcagtgtattcgctgcccaacctattcggcgaaacacccgccgtgcaggtatcgaccaaccgacaagccatcatcatcaacgaacagactcaattcaaagtggatgccgcgctgaaaaacgcaggtattcagaccgacgggatgtttgttgtggacaattcactgaaagtgcgtttcaaagacacagaaacgcagcttaaagcgcgcgacgtcatcgaaaacactttgggcgaagggtatattaccgcgctcaacctgttggcggacagccccgaatggatggcgaaaatcaaagccaatccgatgtttttgggtttggacctgcgcggcggcgtgcatttcaccatgcaggtcgatatgaaagcggcgatgcagaaaacgtttgaacgttattcgggcgacatccgccgcgaactgcgccgcgaaaaaatccgcagcggcacggtgcgtcaggctggaaacagcctgaccgtccctttgcaggatgcaggtgatgtgcaaaaggctctgccgcagttgcgcaagctgtttcctgaagcaacgctgaattcagacggcagcaatatcgtcttgacgctttcggaagaggcggtcaataaagtgtgttccgatgcggtcaaacagaacatcactaccctgcacaaccgtgtgaacgagttgggcgtggccgagcccgtcatccagcagtccggtgcagaccgtatcgtcgtgcagcttccgggcgttcaggatactgccaaggcaaaagacatcatcggccgtaccgcgactttggaattgcgtatggtggaggacgatcctgccaagttgcgcgaggcattggaaggcaacgtgccgagcggttatgagctgctttcaagcggcggagatcgtcccgaaattctgctgatcagcaaacaggtcgagctgacgggcgacaacatcaacgatgcgcaaccgagtttcgaccaaatgggcgcacctgccgtcagtctgagcttggacagcgcgggcggcagcattttcggcgaactgactgccgcaaatgtcggcaaacgcatggcgatggttttgatcgaccaaggaaaatccgaggttgtaaccgcgccggttatccgtactgccattaccggcggacgcgtggaaatttccggaagcatgacgacagccgaagccaatgatacgtctttgctgttgcgtgccggttctcttgccgcaccgatgcagattgtcgaagaacgtaccatcggtccgtctttgggtaaggagaacatcgaaaaaggcttccattcgactttatggggttttgccatcgttgctgcattcatggtggtttactatcgtctgatgggtttcttttctaccattgcattgagtgccaacatactgttcctaatcggtattttgtctgccatgcaggcaacgttgacgttaccgggtatggccgcgctggcgttgactttgggtatggcaatcgactccaacgtcttgattaacgaacgtatccgcgaagaattgcgtgccggcgtgccgccgcagcaggcaatcaatctcggtttccaacacgcatgggcgaccattgtcgattcgaacctgacttcgctgattgccggtatcgcgcttttggtattcggttccggcccggtacgcggttttgcggtcgtacactgtttgggtattctgacttcgatgtattcatccgtcgtcgtattccgtgcgttggtcaatctgtggtacggacgcagacgcaaattgcagaatatttccattggttcggtgtggaagccgaaagccgaaatggcaggaggcaaggagtaa

SEQ ID NO: 14 - protein, 618 aa, Neisseria meningitidis
MMNRYPLWKYLLIVFTIAVAAVYSLPNLFGETPAVQVSTNRQAIIINEQTQFKVDAALKNAGIQTDGMFVVDNSLKVRFKDTETQLKARDVIENTLGEGYITALNLLADSPEWMAKIKANPMFLGLDLRGGVHFTMQVDMKAAMQKTFERYSGDIRRELRREKIRSGTVRQAGNSLTVPLQDAGDVQKALPQLRKLFPEATLNSDGSNIVLTLSEEAVNKVCSDAVKQNITTLHNRVNELGVAEPVIQQSGADRIVVQLPGVQDTAKAKDIIGRTATLELRMVEDDPAKLREALEGNVPSGYELLSSGGDRPEILLISKQVELTGDNINDAQPSFDQMGAPAVSLSLDSAGGSIFGELTAANVGKRMAMVLIDQGKSEVVTAPVIRTAITGGRVEISGSMTTAEANDTSLLLRAGSLAAPMQIVEERTIGPSLGKENIEKGFHSTLWGFAIVAAFMVVYYRLMGFFSTIALSANILFLIGILSAMQATLTLPGMAALALTLGMAIDSNVLINERIREELRAGVPPQQAINLGFQHAWATIVDSNLTSLIAGIALLVFGSGPVRGFAVVHCLGILTSMYSSVVVFRALVNLWYGRRRKLQNISIGSVWKPKAEMAGGKE

SEQ ID NO: 15 - DNA, 1140 bp, Neisseria meningitidis
  CDS (1)..(1140)
atgaaaaaaacactggtggcggcggcaatcctgagcctcgccttgactgcgtgcggcggcggaagcgataccgccgcccaaaccccctccgccaagcccgaagccgaacaatcgggcaaactcaacatctacaactggtcggattatgtcgatcccgaaaccgttgccgcctttgaaaaagaaaccggcatcaagacgcgttccgattattacgacagcaacgaaacactggaggcaaaagtcctgaccggcaaatccggctacgacctgaccgcgccgtccatcgccaacgtcggccggcaaatcaaagcgggcgcgtatcagaaaatcgacaaggcgcaaatcccccattacggcaacatcgataaagatttgctgaaaatgatggaagccgtcgatccgggcaacgaatacgccgtcccctatttctggggcatcaataccttggcaatcaatacccagcaggtgaaaaaagcattgggtacggacaagctgcccgaaaacgaatgggatttggtgttcaaacccgaatacaccgccaaactcaaatcctgcggcatcagctatttcgacagcgcaatcgaacagattcccttggcgttgcactatttgggcaaagaccccaacagtgagaatcccgaagacatcaaagccgccgtcgatatgatgaaagccgtccggggcgacgtgaaacgcttcagctcttccggctatatcgacgatatggcggcgggcaacctgtgtgccgccatcggttacggcggcgatttgaacattgccaaaacccgtgccgaagaagccgcaaacggcgtggaaatcaaagtattgaccccgaaaaccggcgtgggcgtgtgggtggattcctttatgattccgcgcgacgcgcaaaacgttgccaatgcccaccgctatatcgactacacgctccggcccgaggtggcggcgaaaaacggcagcttcgttacctacgcgcccgccagccgtcccgcgcgcgagctgatggatgaaaaatacacctccgacgcatcgattttcccgaacaaagaactgatggaaaaaagtttcatcgtatcgcccaaatccgcagaatccgtcaaactgggcgtgaagctgtggcaagggctcaaagcgggcaaataa

SEQ ID NO: 16 - protein, 379 aa, Neisseria meningitidis
MKKTLVAAAILSLALTACGGGSDTAAQTPSAKPEAEQSGKLNIYNWSDYVDPETVAAFEKETGIKTRSDYYDSNETLEAKVLTGKSGYDLTAPSIANVGRQIKAGAYQKIDKAQIPHYGNIDKDLLKMMEAVDPGNEYAVPYFWGINTLAINTQQVKKALGTDKLPENEWDLVFKPEYTAKLKSCGISYFDSAIEQIPLALHYLGKDPNSENPEDIKAAVDMMKAVRGDVKRFSSSGYIDDMAAGNLCAAIGYGGDLNIAKTRAEEAANGVEIKVLTPKTGVGVWVDSFMIPRDAQNVANAHRYIDYTLRPEVAAKNGSFVTYAPASRPARELMDEKYTSDASIFPNKELMEKSFIVSPKSAESVKLGVKLWQGLKAGK

SEQ ID NO: 17 - DNA, 453 bp, Neisseria meningitidis
  CDS (1)..(453)
atggcaaaagcaatcgaaatcatttcacccaatgaaatttattcagatctgatttttaaggatccggtacctccccatactattgaatataaaatgattaacctagaattgaaagccatcaatctttacgattgtgcctttgaagatttcgttcccgaaatcaaagacaatttttgtgtgggaattgatttagacataggaactgatgatagtgatgctgcagatatattttctgttcgcatatgctctcctaaatggattttgcataattgtttccaaaatcaaagggttaaatggggggcgggtatgatgattatgaatgaatttaatcattctgttattaaatcggaaattgagaaaattcttaaagaatgttcaaaagaaacttgggaaaagtcattgacttatttacttcgttttttttcgtgggaatttgaagattatcagtgctaa

SEQ ID NO: 18 - protein, 150 aa, Neisseria meningitidis
MAKAIEIISPNEIYSDLIFKDPVPPHTIEYKMINLELKAINLYDCAFEDFVPEIKDNFCVGIDLDIGTDDSDAADIFSVRICSPKWILHNCFQNQRVKWGAGMMIMNEFNHSVIKSEIEKILKECSKETWEKSLTYLLRFFSWEFEDYQC

SEQ ID NO: 19 - DNA, 324 bp, Neisseria meningitidis
  CDS (1)..(324)
atgaaaaaatgtattttgggcattttgaccgcgtgtgccgccatgcctgcatttgccgacagaatcggcgatttggaagcacgtctggcgcagttggaacaccgtgtcgccgtattggaaagcggcggcaataccgtcaaaatcgaccttttcggttcaaattccaccatgtatgtatgcagcgttacgccttttcagaagacgtttgaggcaagcgatcggaatgaaggcgtggcgcggcagaaagtgcgtcaggcgtgcaaccgcgaaacttcggcaatgttttgcgaagatgaggcaatccgatgcagaaaattcgattga

SEQ ID NO: 20 - protein, 107 aa, Neisseria meningitidis
MKKCILGILTACAAMPAFADRIGDLEARLAQLEHRVAVLESGGNTVKIDLFGSNSTMYVCSVTPFQKTFEASDRNEGVARQKVRQACNRETSAMFCEDEAIRCRKFD

SEQ ID NO: 21 - DNA, 519 bp, Neisseria meningitidis
  CDS (1)..(519)
atggcaaaacatgaaattgaagaacgcggtgacggtctgattgaaaagatggtcgctgttaatcgcgtaactaaagtagttaaaggtggccgtatcatggctttctcagcactgactgttgttggtgatggtgatggtcgcattggtatgggcaaaggtaaatcaaaagaagtaccagttgctgttcaaaaagcaatggatcaagctcgacgctctatgattaaagtacctttgaaaaacggtactattcatcatgaggttattggccgtcatggtgctactaaagtatttatgcagcctgctaaagagggtagtggcgtaaaagccggtggacctatgcgtttggtttttgatgctatgggcattcataatatctccgccaaagtgcacggatctactaacccatataatatcgtacgtgcaacattagatggtttgtctaagttgcatactcctgctgatatcgcagccaaacgtggcttgacagtggaagacattttgggagttaaccatggctga

SEQ ID NO: 22 - protein, 172 aa, Neisseria meningitidis
MAKHEIEERGDGLIEKMVAVNRVTKVVKGGRIMAFSALTVVGDGDGRIGMGKGKSKEVPVAVQKAMDQARRSMIKVPLKNGTIHHEVIGRHGATKVFMQPAKEGSGVKAGGPMRLVFDAMGIHNISAKVHGSTNPYNIVRATLDGLSKLHTPADIAAKRGLTVEDILGVNHG

SEQ ID NO: 23 - DNA, 2028 bp, Neisseria meningitidis
  CDS (1)..(2028)
ttgtccctgtctgaatttatagaacgccgaacgtcatttaatccgatggttattttgacgactttgttttttgtgtgtgttttggtggtattggttttaaccgtgccggatcaggtgcagatgtggctcgatcgggcaaaagaagtcatttttaccgagttcagctggttttatgttttaacgttttccatttttctgggtttcctgctgatactctcggtcagcagtttgggaaacatcaggctcggacgggatgaagatgtgccggaattcggcttcctgtcgtggctggcgatgctgtttgcggccgggatgggcgtgggtctgatgtttttcggcgtggcagagccgttgatgcattatttttcggacattacggccggcacgccggaacacaggcagcagcaggcattgctgcacacggtgttccattggggcgttcacgcttggtcggtgtacggtacgattgcattggctttggcttatttcggtttccgctacaagctgccgcttgccctgcgttcttgtttttaccccctgttgaaagaaaaaatttccggaaggttcggcgatgccattgatattatggcgttgcttgctacttttttcggcatcatcaccacattggggttcggggcttcgcaactgggcgccggattgcaggaaatgggctggattgccgaaaacagcttcagcgtgcaggttttgattatcgccgccgtcatgtccctcgccgtcgtttcggcaatatccggcgtggggaagggcgtgaaggtgttgagcgagttgaacctgggccttgcgtttttgctgctgttttttgttttggcggcgggacccactgtttacctgttgtcggcattcggcgacaacatagggaactacctcggaaatctggtgcgcctcagttttaaaacttatgcgtacgaacgggaacacaagccgtggtttgaatcttggacggtgctttattgggcgtggtggtgttcttgggcgccgtttgtgggtttgtttatcgcgcgcatttcaaaggggcgcaccatccgcgagtttgtcttcggggttttgctcatccccggcctgttcggcgttttgtggtttaccgtcttcggcaatacggcgatttggctgaatgacggggttgcggggggaatgctcgaaaagatgacctcctctccggaaacgctgctttttaaattctttaattacctccccctgcccgaattgacgagcatcgtcagcctgctggtcatttctctgttttttgtaacttctgccgattccgggatttatgtcctgaacaatattacctctcgggacaaaggcttgagcgcgccacggtggcaggcggttatgtggggcgtgctgatgtctgccgttgccgttttgctgatgcgctcgggcggactcggcaacctgcagtctatgaccctgattgtttccctgccgtttgccctgctgatgctgataatgtgtttcagcctgtggaaaggcttgagtgcggataagaaatattttgagacccgggttaaccctaccagtgtattttggacgggcggcaagtggaaagaacggctggtgcagataatgagccagacgcaggagcaggatattttaaaattcctcaaacagactgcatcgcccgctatgcacgagttgcaacgggagctttcggaagaatacggcttgagcgtccgggtcgataaaatgtttcatcgggacgagcccgcaatcgagttcgtcattcggaaagagacgatgcgcgattttatgtacgggattaagtctgtcgggcaggatgtatccgaccagttgattaacgacggcaagctgccgcatatccggcatcagacaacttacaaaccctacgcttattttttcgacgggcgcgtcgggtacgatgtgcagtatatgaacaaggacgagctgattgccgacattttgaaaaactacgaacgttatttgatgttgttggatgatgtcggtcaggaactgatggcgcacgagcaggtggaattggcagagtaa

SEQ ID NO: 24 - protein, 675 aa, Neisseria meningitidis
LSLSEFIERRTSFNPMVILTTLFFVCVLVVLVLTVPDQVQMWLDRAKEVIFTEFSWFYVLTFSIFLGFLLILSVSSLGNIRLGRDEDVPEFGFLSWLAMLFAAGMGVGLMFFGVAEPLMHYFSDITAGTPEHRQQQALLHTVFHWGVHAWSVYGTIALALAYFGFRYKLPLALRSCFYPLLKEKISGRFGDAIDIMALLATFFGIITTLGFGASQLGAGLQEMGWIAENSFSVQVLIIAAVMSLAVVSAISGVGKGVKVLSELNLGLAFLLLFFVLAAGPTVYLLSAFGDNIGNYLGNLVRLSFKTYAYEREHKPWFESWTVLYWAWWCSWAPFVGLFIARISKGRTIREFVFGVLLIPGLFGVLWFTVFGNTAIWLNDGVAGGMLEKMTSSPETLLFKFFNYLPLPELTSIVSLLVISLFFVTSADSGIYVLNNITSRDKGLSAPRWQAVMWGVLMSAVAVLLMRSGGLGNLQSMTLIVSLPFALLMLIMCFSLWKGLSADKKYFETRVNPTSVFWTGGKWKERLVQIMSQTQEQDILKFLKQTASPAMHELQRELSEEYGLSVRVDKMFHRDEPAIEFVIRKETMRDFMYGIKSVGQDVSDQLINDGKLPHIRHQTTYKPYAYFFDGRVGYDVQYMNKDELIADILKNYERYLMLLDDVGQELMAHEQVELAE

SEQ ID NO: 25 - DNA, 861 bp, Neisseria meningitidis
  CDS (1)..(861)
atgaaaccctattccgccaatcccaacctgaccgaaccgcgccggctgcgcgtgttgctgattgccgccgcgctgggctttctgctgctgatgctggtcgtgccgctcgtcgccgtgttttacgaagccttaaaaggcggttgggatttgtacctgaaatccttaaacgatcccgaagcgtggtctgccatcaaattgacgctgattaccgcgctgattgtcgttcccgtcaatgccgtattgggtgtggcgatggcgtggctgctgacccgttttgattttcgcggcaagcagttgctgaccaccctgctcgatttgccgttttccgtatcgcccgtggtggccggtttgatgttcgtcttattgttcggcgcgcatacggcattgggtggctggctcgaagcgcaaggcatacagattatcttcgccatccccggtattgttttggcgacgctgttcgttaccttcccctttgtcgcacgcgaaatcatcccgctgatgcaggcacagggcgacagcgaagaacaggcggcattgatactcggcgcaagcggctggcagatgttttggcgcgttaccctgcccaacatcaaatgggcgttactctacggcatcatcctcaccaacgcccgcgcgatgggcgagttcggcgcggtcagcgtggtatcgggacacatacgcggcgaaaccaacaccgtcccgcttttggtcgaaatcttctacaacgaatacaacttcaccggcgcattcgccctctccggcgtattggcacttttggcactggcgacgctggcggtgcagaacatcattaccaaattacaagacaaaaaactcgccgccgccgaaaggaatgcaatatga

SEQ ID NO: 26 - protein, 286 aa, Neisseria meningitidis
MKPYSANPNLTEPRRLRVLLIAAALGFLLLMLVVPLVAVFYEALKGGWDLYLKSLNDPEAWSAIKLTLITALIVVPVNAVLGVAMAWLLTRFDFRGKQLLTTLLDLPFSVSPVVAGLMFVLLFGAHTALGGWLEAQGIQIIFAIPGIVLATLFVTFPFVAREIIPLMQAQGDSEEQAALILGASGWQMFWRVTLPNIKWALLYGIILTNARAMGEFGAVSVVSGHIRGETNTVPLLVEIFYNEYNFTGAFALSGVLALLALATLAVQNIITKLQDKKLAAAERNAI

SEQ ID NO: 27 - DNA, 495 bp, Neisseria meningitidis
  CDS (1)..(495)
atgaaaaaaaccctgttggcaattgttgccgtttccgccttaagtgcctgccggcaggcggaagagggaccgccgcctttaccccggcagattagcgaccgttcggtcggacactattgcagtatgaacctgaccgaacacaacggccccaaagcccagattttcttgaacggcaaacccgatcagcccgtttggttctccaccatcaagcagatgttcggctataccaagctgcccgaagagcctaaaggcatccgcgtgatttacgttaccgatatgggcaatgttaccgattggacgaatcccaatgccgacacggagtggatggatgcgaaaaaagccttttacgtcatcgacagcggctttatcggcggtatgggtgcggaagacgcgctgccgttcggcaacaaagagcaggctgagaaatttgcaaaggataaaggcggtaaggttgtcggtttcgacgatatgcctgatacctatattttcaaataa

SEQ ID NO: 28 - protein, 164 aa, Neisseria meningitidis
MKKTLLAIVAVSALSACRQAEEGPPPLPRQISDRSVGHYCSMNLTEHNGPKAQIFLNGKPDQPVWFSTIKQMFGYTKLPEEPKGIRVIYVTDMGNVTDWTNPNADTEWMDAKKAFYVIDSGFIGGMGAEDALPFGNKEQAEKFAKDKGGKVVGFDDMPDTYIFK

SEQ ID NO: 29 - DNA, 810 bp, Neisseria meningitidis
  CDS (1)..(810)
atgagaccatatgctactactatttatcaactttttattttgtttattgggagtgtttttactatgacctcatgtgaacctgtgaatgaaaagacagatcaaaaagcagtaagtgcgcaacaggctaaagaacaaaccagtttcaacaatcccgagccaatgacaggatttgaacatacggttacatttgattttcagggcaccaaaatggttatcccctatggctatcttgcacggtatacgcaagacaatgccacaaaatggctttccgacacgccagggcaggatgcttactccattaatttgatagagattagcgtctattacaaaaaaaccgaccaaggctgggtgctcgaaccatacaaccagcaaaacaaagcgcactttatccaatttctacgcgacggtttggatagcgtggacgatattgttatccgaaaagatgcgtgtagtttaagcacgactatgggagaaagattgcttacttacggggttaaaaaaatgccatctgcctatcctgaatacgaggcttatgaagataaaagacatattcctgaaaatccatattttcatgaattttactatattaaaaaaggagaaaatccggcgattattactcatcggaactatcataggtatggagagaacgattacagcactagcgtaggttcctgtattaacggtttcacggtacggtattacccgtttattcgggaaaagcagcagctcacacagcaggagttggtaggttatcaccaacaagtagagcaattggtacagagttttgtaaacaatccaagtaaaaaataa

SEQ ID NO: 30 - protein, 269 aa, Neisseria meningitidis
MRPYATTIYQLFILFIGSVFTMTSCEPVNEKTDQKAVSAQQAKEQTSFNNPEPMTGFEHTVTFDFQGTKMVIPYGYLARYTQDNATKWLSDTPGQDAYSINLIEISVYYKKTDQGWVLEPYNQQNKAHFIQFLRDGLDSVDDIVIRKDACSLSTTMGERLLTYGVKKMPSAYPEYEAYEDKRHIPENPYFHEFYYIKKGENPAIITHRNYHRYGENDYSTSVGSCINGFTVRYYPFIREKQQLTQQELVGYHQQVEQLVQSFVNNPSKK

SEQ ID NO: 31 - DNA, 285 bp, Neisseria meningitidis
  CDS (1)..(285)
atgaagcaagtcaagaagtcgtcttattttaaatatcaaaaaaggaaaaaaacgatgaacatcgttaaaaaatacgctgtaaaagcagccttggcagccggtatcttcacaccggccattgttatggcagatacctttgatccatccgcgattggtacgcaagtagcgaatgtaatcatgggtttcgtgtcaatggtttccgccgtgggtatggcggccattaccgtgattcttgcaatccaaggcttcaaaatggcttggagcatgattaaatctgtcaaataa

SEQ ID NO: 32 - protein, 94 aa, Neisseria meningitidis
MKQVKKSSYFKYQKRKKTMNIVKKYAVKAALAAGIFTPAIVMADTFDPSAIGTQVANVIMGFVSMVSAVGMAAITVILAIQGFKMAWSMIKSVK

SEQ ID NO: 33 - DNA, 966 bp, Neisseria meningitidis
  CDS (1)..(966)
gtgaaaccgcgtttttattgggcagcctgcgccgtcctgctgaccgcctgttcgcccgaacctgccgccgaaaaaactgtatccgccgcatccgcatctgccgccacgctgaccgtgccgaccgcgcggggcgatgccgttgtgccgaagaatcccgaacgcgtcgccgtgtacgactgggcggcgttggatacgctgaccgaattgggcgtgaatgtgggcgcaaccaccgcgccggtgcgcgtggattatttgcagcctgcatttgacaaggcggcaacggtggggacgctgttcgagcccgattacgaagccctgcaccgctacaatcctcagcttgtcattaccggcgggccgggcgcggaagcgtatgaacagttagcgaaaaacgcgaccaccatagatctgacggtggacaacggcaatatccgcaccagcggcgaaaagcagatggagaccttggcgcggattttcggcaaggaagcgcgcgcggcggaattgaaggcgcagattgacgcgctgttcgcccaaacgcgcgaagccgccaaaggcaaaggacgcgggctggtgctgtcggttacgggcaacaaggtgtccgccttcggcacgcagtcgcggttggcaagttggatacacggcgacatcggcctaccgcctgtagacgaatctttacgcaacgaggggcacgggcagcctgtttccttcgaatacatcaaagagaaaaaccccgattggattttcatcatcgaccgtaccgccgccatcgggcaggaagggccggcggctgtcgaagtattggataacgcgctggtacgcggcacgaacgcttggaagcgcaagcaaatcatcgtcatgcctgccgcgaactacattgtcgcgggcggcgcgcggcagttgattcaggcggcggagcagttgaaggcggcgtttaaaaaggcagaacccgttgcggcggggaaaaagtag

SEQ ID NO: 34 - protein, 321 aa, Neisseria meningitidis
VKPRFYWAACAVLLTACSPEPAAEKTVSAASASAATLTVPTARGDAVVPKNPERVAVYDWAALDTLTELGVNVGATTAPVRVDYLQPAFDKAATVGTLFEPDYEALHRYNPQLVITGGPGAEAYEQLAKNATTIDLTVDNGNIRTSGEKQMETLARIFGKEARAAELKAQIDALFAQTREAAKGKGRGLVLSVTGNKVSAFGTQSRLASWIHGDIGLPPVDESLRNEGHGQPVSFEYIKEKNPDWIFIIDRTAAIGQEGPAAVEVLDNALVRGTNAWKRKQIIVMPAANYIVAGGARQLIQAAEQLKAAFKKAEPVAAGKK

SEQ ID NO: 35 - DNA, 24 bp, Artificial Sequence
  Description of Artificial Sequence Synthetic Oligonucleotide
atgcgaaaaaaacttaccgccctc

SEQ ID NO: 36 - DNA, 18 bp, Artificial Sequence
  Description of Artificial Sequence Synthetic Oligonucleotide
gaatttgtggcgcaaacc

SEQ ID NO: 37 - DNA, 24 bp, Artificial Sequence
  Description of Artificial Sequence Synthetic Oligonucleotide
gaggaagaaaatcattgccgcgac

SEQ ID NO: 38 - DNA, 20 bp, Artificial Sequence
  Description of Artificial Sequence Synthetic Oligonucleotide
atggtgtccgtattcgccgc

SEQ ID NO: 39 - DNA, 24 bp, Artificial Sequence
  Description of Artificial Sequence Synthetic Oligonucleotide
atggctttttatgcttttaaggcg

SEQ ID NO: 40 - DNA, 24 bp, Artificial Sequence
  Description of Artificial Sequence Synthetic Oligonucleotide
tttcgcttcagaagcaggtttggc

SEQ ID NO: 41 - DNA, 21 bp, Artificial Sequence
  Description of Artificial Sequence Synthetic Oligonucleotide
tttgcccgctttgagcccttg

SEQ ID NO: 42 - DNA, 24 bp, Artificial Sequence
  Description of Artificial Sequence Synthetic Oligonucleotide
atgctcaacatctacaactggtcg

SEQ ID NO: 43 - DNA, 21 bp, Artificial Sequence
  Description of Artificial Sequence Synthetic Oligonucleotide
ggagtcggcaaaaaggtgggc

SEQ ID NO: 44 - DNA, 22 bp, Artificial Sequence
  Description of Artificial Sequence Synthetic Oligonucleotide
atgctctcctcacagtctgccc

SEQ ID NO: 45 - DNA, 25 bp, Artificial Sequence
  Description of Artificial Sequence Synthetic Oligonucleotide
atggaactctttaaaatcaaacgcg

SEQ ID NO: 46 - DNA, 25 bp, Artificial Sequence
  Description of Artificial Sequence Synthetic Oligonucleotide
aaccacgatttcttctttcttcttc

SEQ ID NO: 47 - DNA, 20 bp, Artificial Sequence
  Description of Artificial Sequence Synthetic Oligonucleotide
atgagaccatatgctactac

SEQ ID NO: 48 - DNA, 21 bp, Artificial Sequence
  Description of Artificial Sequence Synthetic Oligonucleotide
ttttttacttggattgtttac

(19) United States
(12) Patent Application Publication
(10) Pub. No.: US 2004/0073000 A1
(43) Pub. Date: Apr. 15, 2004

(54) Genes and proteins, and their uses

(76) Inventors: Jonathan Douglas Lane, Berkshire (GB); Martin John Glenton Hughes, Berkshire (GB); Joseph David Santangelo, Berkshire (GB)

Correspondence Address: Saliwanchik Lloyd & Saliwanchik, Suite A 1, 2421 NW 41st Street, Gainesville, FL, 32606-6669, US

(21) Appl. No.: 10/362,327

(22) PCT Filed: Aug. 21, 2001
(86) PCT No.: PCT/GB01/03759

(30) Foreign Application Priority Data
Aug. 24, 2000 (GB) 0020952.8

(51) Int. Cl. C07K 14/22; G01N 33/554; G01N 33/569; A61K 39/02; C07K 16/12; C07H 21/04

(52) U.S. Cl. 530/350; 424/190.1; 435/69.3; 435/252.3; 435/320.1; 435/7.32; 530/388.4; 536/23.7

(57) ABSTRACT

A series of genes from Neisseria meningitidis are shown to encode products which are targets for immunisation. The identification of these genes therefore allows vaccines to be produced and other therapeutic products.

### FIELD OF THE INVENTION

This invention relates to bacterial genes and proteins, and their uses. More particularly, it relates to their use in therapy, for immunisation and in screening for drugs.

### BACKGROUND OF THE INVENTION

Neisseria meningitidis is a Gram-negative bacterial pathogen that is implicated in septic shock and bacterial meningitis. This bacterium is a leading cause of bacterial meningitis in developed countries, and causes large-scale epidemics in Africa and China. In the UK, Neisseria meningitidis is the leading cause of death in childhood apart from road traffic accidents. The bacterium naturally inhabits the human naso-pharynx and then gains access to the blood stream from where it causes severe septicaemia or meningitis. Although current anti-microbials are effective in eliminating the bacterium from the body, the mortalilty from menigococcal septicaemia remains substantial. It would be desirable to provide means for treating or preventing conditions caused by Neisseria meningitidis, e.g. by immunisation.

### SUMMARY OF THE INVENTION

The present invention is based on the discovery of genes in Neisseria meningitidis, the products of which may be located on the outer surface of the organism, and therefore may be used as targets for immuno-therapy.

According to one aspect of the invention, a peptide is encoded by a gene having any of the nucleotide sequences identified in claim 1, or a homologue or a functional fragment thereof. Such a peptide is suitable for therapeutic or diagnostic use, e.g. when isolated.

According to a second aspect of the invention, a polynucleotide encoding a peptide defined above, may also be useful in therapy or diagnosis.

According to a further aspect of the invention, the peptide or the polynucleotide may be used for screening potential antimicrobial drugs.

A further aspect of the invention is the use of any of the products identified herein, for the treatment or prevention of a condition associated with infection by Neisseria or Gram-negative bacteria.

### DESCRIPTION OF THE INVENTION

The present invention is based on the discovery of genes encoding peptides which are located on the cell surface of Neisseria, and which are therefore useful for the preparation of therapeutic agents to treat infection. It should be understood that references to therapy also include preventative treatments, e.g. vaccination. Furthermore, while the products of the invention are intended primarily for the treatment of infections in human patients, veterinary applications are also considered to be within the scope of the invention.

The present invention is described with reference to Neisseria meningitidis. However, all the Neisseria strains, and many other Gram-negative bacterial strains are likely to include related peptides or proteins having amino acid sequence identity or similarity to those identified herein. Organisms likely to contain the peptides include, but are not limited to the genera Salmonella, Enterobacter, Klebsiella, Shigella and Yersinia.

Preferably, the peptides that may be useful in the various aspects of the invention have greater than a 40% similarity with the peptides identified herein. More preferably, the peptides have greater than 60% sequence similarity. Most preferably, the peptides have greater than 80% sequence similarity, e.g. 95% similarity. With regard to the polynucleotide sequences identified herein, related polynucleotides that may be useful in the various aspects of the invention may have greater than 40% identity with the sequences identified herein. More preferably, the polynucleotide sequences have greater than 60% sequence identity. Most preferably, the polynucleotide sequences have greater than 80% sequence identity, e.g. 95% identity.

The terms “similarity” and “identity” are known in the art. The use of the term “identity” refers to a sequence comparison based on identical matches between correspondingly identical positions in the sequences being compared. The term “similarity” refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity.

Levels of identity between gene sequences and levels of identity or similarity between amino acid sequences can be calculated using known methods. In relation to the present invention, publicly available computer based methods for determining identity and similarity include the BLASTP, BLASTN and FASTA (Atschul et al., J. Molec. Biol., 1990; 215:403-410), the BLASTX program available from NCBI, and the Gap program from Genetics Computer Group, Madison Wis. The levels of similarity and identity provided herein, were obtained using the Gap program, with a Gap penalty of 12 and a Gap length penalty of 4 for determining the amino acid sequence comparisons, and a Gap penalty of 50 and a Gap length penalty of 3 for the polynucleotide sequence comparisons.

Having characterised a gene according to the invention, it is possible to use the gene sequence to search for related genes or peptides in other microorganisms. This may be carried out by searching in existing databases, e.g. EMBL or GenBank.

The techniques mentioned herein are well known in the art. However, reference is made in particular to Sambrook et al, Molecular Cloning, A Laboratory Manual (1989) and Ausubel et al., current Protocols in Molecular Biology (1995), John Wiley & Sons, Inc.

Peptides or proteins according to the invention may be purified and isolated by methods known in the art. In particular, having identified the gene sequence, it will be possible to use recombinant techniques to express the genes in a suitable host. Active fragments and related molecules can be identified and may be useful in therapy. For example, the peptides or their active fragments may be used as antigenic determinants in a vaccine, to elicit an immune response. They may also be used in the preparation of antibodies, for passive immunisation, or diagnostic applications. Suitable antibodies include monoclonal antibodies, or fragments thereof, including single chain Fv fragments. Humanised antibodies are also within the scope of the invention. Methods for the preparation of antibodies will be apparent to those skilled in the art.

Active fragments of the peptides are those that retain the biological function of the peptide. For example, when used to elicit an immune response, the fragment will be of sufficient size, such that antibodies generated from the fragment will discriminate between that peptide and other peptides on the bacterial microorganism. Typically, the fragment will be at least 30 nucleotides (10 amino acids) in size, preferably 60 nucleotides (20 amino acids) and most preferably greater than 90 nucleotides (30 amino acids) in size.

It should also be understood, that in addition to related molecules from other microorganisms, the invention encompasses modifications made to the peptides and polynucleotides identified herein which do not significantly alter the biological function. It will be apparent to the skilled person that the degeneracy of the genetic code can result in polynucleotides with minor base changes from those specified herein, but which nevertheless encode the same peptides. Complementary polynucleotides are also within the invention. Conservative replacements at the amino acid level are also envisaged, i.e. different acidic or basic amino acids may be substituted without substantial loss of function.

The preparation of vaccines based on the identified peptides will be known to those skilled in the art. Vaccine compositions can be formulated with suitable carriers or adjuvants, e.g. alum, as necessary or desired, to provide effective immunisation against infection. The preparation of vaccine formulations will be apparent to the skilled person.

More generally, and as is well known to those skilled in the art, a suitable amount of an active component of the invention can be selected, for therapeutic use, as can suitable carriers or excipients, and routes of administration. These factors would be chosen or determined according to known criteria such as the nature/severity of the condition to be treated, the type and/or health of the subject etc.

In a separate embodiment, the products of the invention may be used in screening assays for the identification of potential antimicrobial drugs or for the detection for virulence. Routine screening assays are known to those skilled in the art, and can be adapted using the products of the invention in the appropriate way. For example, the products of the invention may be used as the target for a potential drug, with the ability of the drug to inactivate or bind to the target indicating its potential antimicrobial activity.

The genes of the invention may also be implicated in the virulence of the microorganism, and therefore deleting or inactivating the gene may be sufficient to produce an attenuated (avirulent) microorganism.

The attenuated microorganisms may be prepared with a mutation that disrupts the expression of any of the genes identified herein. The skilled person will be aware of methods for disrupting expression of particular genes. Techniques that may be used include insertional inactivation or gene deletion techniques. Attenuated microorganisms according to the invention may also comprise additional mutations in other genes, for example in a second gene identified herein or in a separate gene required for growth of the microorganism, e.g. an aro mutation or, with regard to Salmonella, in a gene located in the SPI2 region identified in WO-A-96/17951.

Attenuated microorganisms may also be used as carrier systems for the delivery of heterologous antigens, therapeutic proteins or nucleic acids (DNA or RNA). In this embodiment, the attenuated microorganisms are used to deliver a heterologous antigen, protein or nucleic acid to a particular site in vivo. Introduction of a heterologous antigen, peptide or nucleic acid into an attenuated microorganism can be carried out by conventional techniques, including the use of recombinant constructs, e.g. vectors, which comprise polynucleotides that express the heterologous antigen or therapeutic protein, and also include suitable promoter sequences. Alternatively, the gene that encodes the heterologous antigen or protein may be incorporated into the genome of the organism and the endogenous promoters used to control expression.

The various products of the invention may also be used in veterinary applications.

The peptides of the present invention were identified as follows:

Identification of Peptides

A partial gene library of Neisseria meningitidis (strain C311+) chromosomal DNA was prepared using the plasmid vectors pFW-phoA1, pFW-phoA2 and pFW-phoA3 (Podbielski, A. et al., Gene 1996; 177:137-147). These plasmids possess a constitutive spectinomycin adenyltransferase antibiotic resistance marker, which confers a high level of spectinomycin resistance and is therefore easily selected. Furthermore, these vectors contain a truncated (leaderless) Escherichia coli phoA gene for alkaline phosphatase. The three vectors differ only with respect to the reading frame in which the leaderless phoA gene exists, as compared to an upstream in-frame BamHI restriction enzyme site. Because this truncated E. coli phoA gene lacks the appropriate leader sequence for export of this enzyme across the bacterial membrane, extracellular alkaline phosphatase activity is absent when these plasmids are propagated in an E. coli phoA mutant (e.g. strain DH5α). The chromogenic alkaline phosphatase substrate, XP (5-Bromo-4-chloro-3-indolyl-phosphate), does not enter intact bacterial cells and therefore only exported or surface-associated alkaline phosphatase activity can be detected. When exported or surface-associated alkaline phosphatase activity is present, the chromogenic XP substrate is cleaved to yield a blue pigment and the corresponding bacterial colonies can be identified by their blue colour.

Plasmid DNA was digested to completion with BamHI and dephosphorylated using shrimp alkaline phosphatase. Neisseria genomic DNA was partially digested with Sau3AI, such that a majority of fragments appeared to be 0.5-1.0 kb in size when observed as bands on a 1% agarose gel stained with ethidium bromide. These Sau3AI fragments were ligated into the prepared pFW-phoA vectors. E. coli strain DH5α was chosen as the cloning host since it lacks a functional phoA gene. Recombinant plasmids were selected on Luria agar containing 100 μg/ml of spectinomycin and 40 μg/ml of the chromogenic XP substrate. E. coli transformants harbouring plasmids containing Neisseria meningitidis insert DNA that complements the export signal sequence of the leaderless phoA gene were identified by the blue colour of the colonies.

Neisseria meningitidis insert DNA that complemented the export signal sequence of the leaderless phoA gene was sequenced and the resulting sequence was searched for known proteins in the GenBank database. The results are shown in Table 1.

TABLE 1

| SEQ ID NO. | | Ref. | | Accession |
|---|---|---|---|---|
| Gene | Protein | Name | Putative Protein | No. |
| 1 | 2 | pho1-94 | L-lactate permease | NMB0543 |
| 3 | 4 | pho1-61 | Membrane fusion protein | NMB1716 |
| 5 | 6 | pho1-96 | Protein-export membrane protein | NMB0608 |
| | | | SECF | |
| 7 | 8 | pho2-7 | Organic solvent tolerance | NMB0280 |
| | | | protein | |
| 9 | 10 | pho2-10 | Penicillin-binding protein 4 | NMB0749 |
| 11 | 12 | pho2-6 | Thiol:disulphide interchange | NMB1519 |
| | | | protein DSBD | |
| 13 | 14 | pho2-35 | Protein-export membrane protein | NMBO607 |
| | | | SECD | |
| 15 | 16 | pho2-66 | Spermidine/Putrescine-binding | NMB0623 |
| | | | protein | |
| 17 | 18 | pho2-76 | Hypothetical 17.6 kD protein | NMB0350 |
| 19 | 20 | pho2-80 | Hypothetical 11.9 kD protein | NMB0844 |
| 21 | 22 | pho2-81 | — | NMB0159 |
| 23 | 24 | pho2-86 | — | NMB1277 |
| 25 | 26 | pho2-90 | Sulphate ABC transporter | NMB0880 |
| 27 | 28 | pho2-91 | — | NMB0580 |
| 29 | 30 | pho2-5 | FRPC operon protein | NMB1412 |
| | | | | NMB0584 |
| | | | | NMB0364 |
| | | | | NMB1414 |
| 31 | 32 | pho2-25 | Hypothetical 10.2 kD protein | NMB1546 |
| | | | | NMB1631 |
| 33 | 34 | pho2-41 | — | NMB1830 |

Protective Properties of Candidate Protein Vaccines

Genes identified in the screen were assessed as potential protein vaccine candidates based on the ability of the cloned, expressed, proteins to raise an immune response in rabbits, with the resulting antibodies having the ability to stimulate complement-mediated bacteriolysis of Neisseria meningitidis. Protective responses were determined by live bacterial challenge of mice immunised with recombinant proteins.

In summary, the candidate genes were PCR amplified, cloned and the encoded protein expressed and purified. The purified protein was used to generate antibodies for use in Enzyme Linked Immuno-Sorbent Assays (ELISA). The PorA gene was also PCR amplified, cloned, expressed and purified. Monoclonal antibodies against PorA have been shown to passively protect animals in an infant rat model of infection (Saukkonen et al., Microb. Pathog., 1987; 3(4): 261-267). Therefore, this protein was used as a positive control in some experiments. PorA has been shown to be unable to protect an animal against challenge from different strains of N. meningitidis (Poolman, Infect. Dis., 1995; 4: 13), therefore any candidate that is able to generate a protective immune response against a diverse range of N. meningitidis strains, offers advantages over PorA.

PCR Amplification of DNA.

Candidate genes were amplified by PCR using genomic DNA from strain MC58 as the DNA template (McGuinness et al, Lancet, 1991; 337:514). The primers are listed in Table 2. F denotes the forward primer and R the reverse primer. The primer pair PRAF and PRAR was used to amplify the PorA gene DNA.

TABLE 2

| Candidate | Primer Sequence | SEQ ID NO. |
|---|---|---|
| PRAF | 5′ATGCGAAAAAAACTTACCGCCCTC | 35 |
| PRAR | 5′GAATTTGTGGCGCAAACC | 36 |
| phoA 1-94R | 5′GAGGAAGAAAATCATTGCCGCGAC | 37 |
| phoA 1-94F | 5′ATGGTGTCCGTATTCGCCGC | 38 |
| phoA 1-61F | 5′ATGGCTTTTTATGCTTTTAAGGCG | 39 |
| phoA 1-61R | 5′TTTCGCTTCAGAAGCAGGTTTGGC | 40 |
| phoA 2-66R | 5′TTTGCCCGCTTTGAGCCCTTG | 41 |
| phoA 2-66F | 5′ATGCTCAACATCTACAACTGGTCG | 42 |
| phoA 2-10R | 5′GGAGTCGGCAAAAAGGTGGGC | 43 |
| phoA 2-10F | 5′ATGCTCTCCTCACAGTCTGCCC | 44 |
| phoA 1-97F | 5′ATGGAACTCTTTAAAATCAAACGCG | 45 |
| phoA 1-97R | 5′AACCACGATTTCTTCTTTCTTCTTC | 46 |
| phoA 2-5F | 5′ATGAGACCATATGCTACTAC | 47 |
| phoA 2-5R | 5′TTTTTTACTTGGATTGTTTAC | 48 |

Cloning of Vaccine Candidates.

PCR amplified DNA from candidates was cloned directly into the InVitrogen pCRT7/CT-TOPO vector. This vector provides a T7 promoter, ribosome binding site and C-terminal 6xHis tag fusion to facilitate expression and purification of recombinant proteins using metal affinity chromatography.

For cloning, the ligation reaction was transformed in TOP10F′ cells (Invitrogen). DNA preparations from transformant DNA clones were screened to check the orientation of the insert DNA. Clones from candidates that appeared to have the insert DNA in the correct orientation were sequenced to confirm the integrity of the 5′ region of the construct.

Expression and Purification.

Cloned candidates were tested for expression of the candidate genes following transformation into HMS174(DE3)pLysS competent cells (Invitrogen). Expression of candidate clones was induced with IPTG and expression analysed by SDS PAGE and western blotting using anti-His antibody after four hours induction. Candidate protein was purified via Talon resin (metal affinity column utilising the 6xHis-tag cloned at the carboxy terminus of the protein (Clontech)) utilizing an imidozole buffer gradient for elution of protein from the resin (10-100 mM).

Antibody Production.

Prior to antibody production, animal serum was pre-screened for low reactogenicity to whole cell Neisseria meningitidis in ELISA assays. Antibodies were raised against each of the cloned and purified candidates in rabbits using 100 μg of proteins for initial vaccination with Freund's adjuvant and three subsequent boosts at 28-day intervals with Freund's incomplete adjuvant. Serum was collected after each boost to generate sera samples.

ELISA Against Whole Heat Killed N. meningitidis

ELISA assays against heat killed N. meningitidis were carried out to confirm that antibodies raised to purified proteins recognise N. meningitidis cells. These assays were carried out on strain MC58 as well as:

Neisseria meningitidis (B) Type 1000

Neisseria meningitidis (B) Type SW2 107

Neisseria meningitidis (B) Type NGH38

Neisseria meningitidis (B) Type NGE28

Neisseria meningitidis (B) Type 2996

These are all prevalent disease-causing strains and span the genetic diversity of this species based on dendrograms generated by MLST (multi-locus sequence typing).

Preparation of Heat Killed N. meningitidis

N. meningitidis was grown on Columbia agar with chocolated horse blood (Oxoid) for 14 hours at 37° C. in 5% CO₂. The cells were scraped from agar plate and resuspended the cells in 20 ml PBS in a 50 ml tube. The cell suspension was heated for 30 minutes at 56° C. to kill the bacteria.

A 50 μl sample of the heat killed N. meningitidis was spread to Columbia agar with chocolated horse blood (Oxoid) and incubated for 18 hours at 37° C., 5% CO₂. This allows confirmation that all N. meningitidis cells have been killed. The heat-killed cells were then washed in PBS. The OD₆₂₀ of the suspension was adjusted to 0.1 OD units versus PBS.

ELISA with Heat Killed N. meningitidis

ELISA assays were carried out using the heat killed whole cell N. meningitidis. ELISA plates were coated overnight with heat-killed cells (50 μl of killed bacteria in PBS to each well of 96 well plate and incubated 4° C.). Standard ELISA protocols were followed, with all incubations at 37° C. for 1 hour. PBS/3% BSA blocking solution, PBS/Tween 0.1% wash solution, anti-rabbitAP conjugate secondary antibody (Sigma) and Sigma Fast P Nitrophenyl phosphate detection reagent (Sigma) were utilised. The data was read at 405 nm using an appropriate micro-titre plate reader. The data was generated using sera available seven days after the first booster vaccination (day 35 after first vaccination).

ELISA Data.

The results showed that the anti-sera raised against each candidate protein elicited a strong response against the different strains of N. meningitidis.

In Vivo Screening.

To evaluate the protective efficacy of vaccine candidates, adult mice were immunised with recombinant proteins and the protective response determined by live bacterial challenge.

For each vaccine candidate, 15 six week old balb/C mice were vaccinated (subcutaneously) with 25 μg of antigen on two separate occasions at three week intervals. One week after the end of the immunisation schedule, the group was challenged with the homologous bacterial strain MC58. The bacteria were inoculated intraperitoneally in a volume of 500 μl in Brain Heart Infusion/0.5% iron dextran media at a dose of 1×10⁶cfu. Previous results have shown that iron is required for initiation of bacteraemic disease in these animals. This model has previously been used to demonstrate the protective efficacy of vaccination (Lissolo et al, Infect. Immun., 1995; 63: 884-890).

Control groups included animals vaccinated with adjuvant alone (negative control), with adjuvant combined with purified PorA (positive control) or an attenuated homologous strain. Survival was monitored following challenge.

Animals vaccinated with the candidate pho2-5 showed 80% (12/15) survival compared to non-vaccinated controls where 13% (2/15) survived. The pho2-5 candidate showed levels of protection equivalent to porA protein (13/15).

Animals vaccinated with candidates pho2-10, pho1-94 or pho2-66 had 40% (6/15), 47% (7/15) or 27% (4/15) survival respectively, compared to the non-vaccinated controls, where 13% (2/15) survived.

1. A peptide encoded by a gene including any of the nucleotide sequences identified herein as SEQ ID NOS. 1, 3, 5, 7, 9, 11, 13, 15, 17, 19, 21, 23, 25, 27, 29, 31 and 33, of N. meningitidis, or a homologue thereof in a Gram-negative bacterium, or a functional fragment thereof, for therapeutic or diagnostic use.

2. A peptide according to claim 1, wherein the homologue has at least 40% sequence similarity or identity at the peptide or nucleotide level.

3. A peptide according to claim 1 or claim 2, wherein the homologue has at least 60% sequence similarity or identity at the peptide or nucleotide level.

4. A peptide according to any preceding claim, wherein the homologue has at least 90% sequence similarity or identity at the peptide or nucleotide level.

5. A peptide according to claim 1, comprising any of the amino acid sequences defined herein as SEQ ID NOS. 2, 4, 6, 8, 10, 12, 14, 16, 18, 20, 22, 24, 26, 28, 30, 32 and 34.

6. A polynucleotide encoding a peptide according to any preceding claim, for therapeutic or diagnostic use.

7. A host transformed to express a peptide according to any of claims 1 to 5.

8. A microorganism comprising a mutation that disrupts the expression of any of the nucleotide sequences defined in claim 1.

9. A microorganism according to claim 8, wherein the mutation is insertional inactivation or a gene deletion.

10. A microorganism according to claim 8 or claim 9, wherein the microorganism is Neisseria meningitidis.

11. A microorganism according to any of claims 8 to 10, comprising a mutation in a second nucleotide sequence.

12. A microorganism according to any of claims 8 to 11, for therapeutic or diagnostic use.

13. A vaccine comprising a peptide according to any of claims 1 to 5, or the means for its expression.

14. An antibody raised against a peptide according to any of claims 1 to 5.

15. Use of a product according to any of claims 1 to 7, in a screening assay.

16. Use of a product according to any of claims 1 to 12, for the manufacture of a medicament for use in the treatment or prevention of a condition associated with infection by Neisseria or Gram-negative bacteria.
